(19) United States
(12) Patent Application Publication
(10) Pub. No.: US 2023/0032171 A1
(43) Pub. Date: Feb. 2, 2023

(54) FILTERING APPARATUS FOR LENS POLISHING WASTEWATER AND SYSTEM FOR REDUCING POLISHING WASTEWATER

(71) Applicant: RODEK CO LTD, Cheongju-si, Chungcheongbuk-do (KR)

(72) Inventor: Byeong Seon EUN, Cheongju-si, Chungcheongbuk-do (KR)

(73) Assignee: RODEK CO LTD, Cheongju-si, Chungcheongbuk-do (KR)

(21) Appl. No.: 17/789,524

(22) PCT Filed: Mar. 31, 2021
(86) PCT No.: PCT/KR2021/003960

§ 371 Date: Jun. 28, 2022

(30) Foreign Application Priority Data
Apr. 1, 2020 (KR) 10-2020-0039648
Oct. 28, 2020 (KR) 10-2020-0141146

(51) Int. Cl. B24B 55/12; B01D 35/02; A61L 9/00; B01D 46/00; B04C 9/00; C02F 1/40; C02F 1/70; C02F 9/00

(52) U.S. Cl. CPC B24B 55/12; B01D 35/02; A61L 9/00; B01D 46/00; B04C 9/00; C02F 1/40; C02F 1/70; C02F 9/00

(57) ABSTRACT

The present invention provides a filtering apparatus for lens polishing wastewater and a system for reducing polishing wastewater, which efficiently remove fine particles such as lens particles and fine lens particles that are mixed into polishing water in a lens cutting and polishing process in a lens polishing machine such as an edger, to reduce the polishing water to be reused in lens polishing, and can prevent water pollution by fundamentally preventing the discharge of polishing water in which fine particles are mixed.

### TECHNICAL FIELD

The present invention relates to an apparatus for treating lens polishing wastewater, and more particularly, to a filtering apparatus for lens polishing wastewater and a system for reducing polishing wastewater, which separate and remove fine particles such as lens particles, fine lens particles, and microplastics from polishing wastewater generated in a lens processing process in a lens polishing machine such as an edger to circulate and reuse the polishing wastewater as polishing water, thereby preventing water pollution by fundamentally preventing water discharge of the polishing wastewater in which the fine particles are mixed and preventing pollution.

### BACKGROUND ART

In general, a process of polishing a lens by using a lens polishing machine may generate extremely large amount of dusts and particles together with heat due to friction between the lens that is an object to be processed and a polishing unit, and the dusts and particles may cause a polishing defect.

Also, a property of the lens or the polishing unit may be changed or damaged by the heat generated during the polishing process.

To solve the above-described problem, polishing water is supplied to a contact portion between the lens and the polishing unit during the lens is processed by using the lens polishing machine to restrict temperature increase of the lens and the lens polishing machine and prevent dusts from blowing.

For example, since a spectacle lens is manufactured into a circular shape, and a glasses frame is designed with various shapes, an edge of the spectacle lens is processed to be fit to a shape and a groove of the glasses frame by using an edger so as to fit the spectacle lens to the glasses frame.

Since the edger generates high temperature heat of 900° C. or more due to friction occurring when a lens such as a spectacle lens made of glass or plastic materials is cut by diamond grindstone rotating at a speed of 3000 rpm to 6000 rpm, polishing water the performs lubrication and cooling for preventing the lens from being damaged during polishing is used, or refrigerant containing an anticorrosive agent and a cleaning agent for preventing foreign substances from being attached to the diamond grindstone to improve a grinding force is used.

Since the fine particles such as lens particles, fine lens particles, and microplastics generated while the spectacle lens is polished in the edger is mixed with the polishing water and discharged in the form of polishing wastewater, the polishing wastewater is collected in a water collecting container and precipitated, and then the polishing wastewater is filtered by using a filter and reused as polishing water. When the filter does not perform filtering because pores of the filter is clogged by sludge produced by agglomeration of fine particles, the polishing wastewater is discharged through a sewer.

However, since most of fine particles contained in the polishing wastewater is not filtered by a filter and not filtered even by a sewage treatment facility, the fine particles flow directly into rivers and seas through a sewer pipe and cause secondary pollution to fish and shellfish and marine ecosystems.

Also, the polishing wastewater contains carcinogenic or suspected substances such as dichloromethane, chloroform, phenol, cyanide, 1.4-dioxin, formaldehyde and heavy metals and has COD that is greater by 6 times to 13 times of a reference value and SS (floating substances) that is greater by 25 times to 21 times of a reference value. Thus, serious water pollution and environmental pollution may occur.

In recent years, the enforcement regulation of the water environment conservation act (amendment on Oct. 17, 2019; enforcement on Jan. 1, 2021; report on Jun. 30, 2021) stipulates to manage the lens polishing wastewater of optical shops within the legal system. Since external discharge (sewer) of the lens polishing wastewater is prohibited from January 2021 according to the amended enforcement regulation of the water environment conservation act, all of domestic 10,500 optical shops are obliged to have legal facilities and voluntarily report the legal facilities to the competent administrative office from January to June 2021.

According to the enforcement regulation of the water environment conservation act, the facility of manufacturing the lens in the optical shop that is classified as manager and installation of other water pollution sources should process the wastewater with a proper method such as filtering to remove water pollution substances when wastewater is discharged by treating the wastewater below the discharge standard, and use a filtering apparatus made of a material such as non-woven fabric with a mean pore size of 10 μm or less or having an equivalent performance. Here, the performance of the filtering apparatus should be properly maintained by frequently removing the generated sludge.

As one of methods for treating lens polishing wastewater of the optical shops, Korean Patent Registration No. 10-0419351 discloses a method and an apparatus for regenerating lens polishing water, which filter foreign substances by a filter while the polishing water is dropped by own weight to a storage tank disposed therebelow by supplying polishing water to the filter tank including the filter at an inner lower portion thereof when the polishing water introduced to the storage tank is cloudy, remove moisture in the foreign substances stacked on the filter, and then remove the foreign substances.

This patent may reduce water consumption through filtering and reuse of the polishing wastewater and effectively filter polishing particles by using a fiber filter for filtering the polishing wastewater. However, this patent exhibits the low economic efficiency and inconvenient maintenance due to inevitable frequent replacement of the filter because the filter is frequently clogged by fine particles contained in the polishing wastewater in case of a large lens process quantity.

To solve the above-described problem, Korean Patent Registration No. 10-1889229 discloses an apparatus for treating polishing wastewater for an edger, which includes: a first filter disposed at an inlet of a water collecting tank for collecting the polishing wastewater discharged from the edger to filter foreign substances each having a diameter of 1 mm or more; a second filter disposed at an outlet of the water collecting tank to filter foreign substances each having a diameter of 1 mm or more; and a sludge filter net disposed in the water collecting tank to filter only sludge, and further include a ultrasonic oscillator disposed below the water collecting tank for preventing clogging of the sludge filter net and improving agglomeration of the sludge by transmitting high frequency vibrations to the collected polishing wastewater.

Since each of the first filter and the second filter is a metal mesh type filter, the filters may be cleaned and then reused. However, since the clogging of the filter still frequently occur in case of the large lens process quantity, the filter causes considerable inconvenience in that the filter is required to be frequently disassembled and cleaned.

Also, since combustion gases and dusts containing carcinogenic or suspected substances are diffused over a working space by high temperature heat caused by friction although polishing water and refrigerant are used during lens processing, health of a worker may be adversely affected by the dusts and the combustion gases in addition to malodor.

Therefore, a filtering apparatus for lens polishing wastewater and a system for reducing polishing wastewater, which may reuse the polishing wastewater as the polishing water by effectively filtering and reducing the polishing wastewater without frequent replacement of a filter and without disassembly and cleaning of a filter, effectively filter and remove fine particles such as lens particles, fine lens particles, and microplastics from the polishing wastewater, and improve a working environment in which malodor and carcinogenic or suspected substances are inhaled.

### DISCLOSURE OF THE INVENTION

### Technical Problem

The present invention is created as a result of continuous research with all efforts to develop a new filtering apparatus for lens polishing wastewater and a system for reducing polishing wastewater, which may minimize consumption of a filter by filtering and removing fine particles such as lens particles, fine lens particles, and microplastics contained in polishing wastewater even without using a mesh-type filter, reduce water consumption by circulating polishing water to be reused, achieve an effect of preventing water pollution and environmental pollution because the polishing wastewater is not discharged at all, based on the inventor's idea of solving limitations and problems of a typical apparatus for treating lens polishing wastewater by comprehensively considering the above-described features.

Thus, the present invention provides a filtering apparatus for lens polishing wastewater and a system for reducing polishing wastewater, which may reuse polishing wastewater as polishing water by filtering and removing fine particles contained in the polishing wastewater without frequent replacement and cleaning of a filter.

The present invention also provides a filtering apparatus for lens polishing wastewater and a system for reducing polishing wastewater, which may create a clean working environment by removing malodor and carcinogenic or suspected substances generated during polishing of a lens.

The present invention also provides a filtering apparatus for lens polishing wastewater and a system for reducing polishing wastewater, which may simply separate and discharge sludge by completely removing moisture contained in filtered sludge.

The present invention also provides a filtering apparatus for lens polishing wastewater and a system for reducing polishing wastewater, which may be used for all purposes regardless of kinds and shapes of lens polishing machines.

The object of the present invention is not limited to the aforesaid, but other objects not described herein will be clearly understood by those skilled in the art from descriptions below.

### Technical Solution

One aspect of the present invention provides a filtering apparatus for lens polishing wastewater, including: a casing having a tapered shape having a diameter that gradually decreases in a direction from an upper portion to a lower portion thereof so as to form a rotary flow while polishing wastewater pumped by a water pump is introduced from the upper portion and discharged to the lower portion; a mesh net having an inverted truncated cone shape such that an outer circumferential surface thereof closely contacts an inner circumferential surface of the casing, and a plurality of valleys are radially formed in an inner circumferential surface thereof; and a non-woven bag mounted to the inside of the mesh net in such a manner that an inlet thereof is fixed to the upper portion of the casing, and an outer circumferential surface thereof closely contacts the inner circumferential surface of the mesh net by the polishing wastewater discharged downward while forming the rotary flow at the upper portion of the casing, the non-woven bag configured to discharge reduced water obtained by filtering and removing floating substances of fine particles contained in the polishing wastewater to the outside.

Thus, the present invention may filter and remove the floating substances of the fine particles such as lens particles, fine lens particles, and microplastics contained in the polishing wastewater and reuse the polishing wastewater as polishing water to prevent water pollution and environmental pollution because the polishing wastewater is not discharged at all.

Another aspect of the present invention provides a system for reducing polishing wastewater, including: the filtering apparatus; and a polishing water tank configured to store polishing wastewater discharged from a lens polishing machine and pump the stored polishing wastewater by a water pump operated according to driving of the lens polishing machine to the filtering apparatus, and the system supplies the reduced water discharged from the filtering apparatus to the lens polishing machine.

In an aspect of the present invention, the system may further include a filter net disposed in the polishing water tank and configured to prevent lens particles having a relatively large particle size and contained in the polishing wastewater discharged from the lens polishing machine from being introduced into the polishing water tank.

In an aspect of the present invention, the system may further include: a branch pipe that is branched from a polishing wastewater pipe installed to supply the polishing wastewater pumped by the water pump to the filtering apparatus and installed on a tank cover of the polishing water tank; and a plurality of spray nozzles mounted to an end of the branch pipe to spray the polishing wastewater supplied through the branch pipe into the polishing water tank from an upper portion thereof in order to remove bubbles in the polishing water tank.

In an aspect of the present invention, the system may further include a plurality of tubes connected with the branch pipe and configured to supply the polishing wastewater to an inner floor of the polishing water tank so as to generate a flow of the polishing wastewater in the polishing water tank and a diffuser mounted to an end of each of the plurality of tubes in order to diffuse the polishing wastewater in all directions.

In an aspect of the present invention, a blocking wall for preventing the polishing wastewater sprayed from the spray nozzle from being splashed and leaked to the outside may be formed on a bottom surface of the tank cover.

In an aspect of the present invention, the system may further include: a deodorizing apparatus configured to suction air in the polishing water tank and discharge the air to the outside after filtering fine particles and ultrafine particles in the air; and an air pocket disposed between the water pump and the filtering apparatus to reduce a pressure of air flowing backward to the water pump from the filtering apparatus due to pressure change when the water pump is stopped.

### Advantageous Effects

According to the embodiment and technical concept of the present invention based on the unique solution method for solving the above technical objects, the polishing wastewater may be reused as the polishing water by effectively filtering and removing the fine particles from the polishing wastewater without frequent replacement or cleaning of the filter for filtering the fine particles contained in the polishing wastewater for a long time.

Thus, the consumption of the filter may be minimized to increase the economic efficiency and exhibit the water saving effect, and water pollution and environmental pollution may be prevented as the polishing wastewater is not discharged at all.

Also, the clean working environment may be provided by removing the malodor and the carcinogenic or suspected substances generated during the polishing of the lens.

Also, the water pollution source may be fundamentally blocked by completely removing the moisture contained in the filtered sludge and conveniently separating and discharging the sludge.

Also, the filtering apparatus for lens polishing wastewater and the system for reducing the polishing wastewater including the same according to the present invention may be used in all sorts of lens polishing machines regardless of the kinds and shapes of the lens polishing machines and improve usability and convenience as the entire system is operated together even when only the lens polishing machine is driven.

Furthermore, as the polishing wastewater is sprayed through the spray nozzle, the bubbles in the polishing water tank may be further completely removed.

In addition, since the natural and smooth flow of the polishing wastewater is continuously generated in the polishing water tank through the tubes and the diffuser, the fine particles mixed into the polishing wastewater may be prevented from being precipitated on the corner or the floor in the polishing water tank.

The effects of the present invention are not limited to the aforementioned effects, but other objects not described herein will be clearly understood by those skilled in the art from descriptions below.

### BRIEF DESCRIPTION OF THE DRAWINGS

FIG. 1 is a schematic block diagram illustrating a system for reducing lens polishing wastewater according to an embodiment of the present invention.

FIG. 2 is a perspective view illustrating the system for reducing the lens polishing wastewater according to an embodiment of the present invention.

FIG. 3 is a front view illustrating the system for reducing the lens polishing wastewater according to an embodiment of the present invention.

FIG. 4 is a rear view illustrating the system for reducing the lens polishing wastewater according to an embodiment of the present invention.

FIG. 5 is a right view illustrating the system for reducing the lens polishing wastewater according to an embodiment of the present invention.

FIG. 6 is a left view illustrating the system for reducing the lens polishing wastewater according to an embodiment of the present invention.

FIG. 7 is an exploded perspective view illustrating the system for reducing the lens polishing wastewater according to an embodiment of the present invention.

FIG. 8 is a cross-sectional view illustrating the system for reducing the lens polishing wastewater according to an embodiment of the present invention.

FIG. 9 is a right view illustrating an air pocket of the system for reducing the lens polishing wastewater according to an embodiment of the present invention.

FIG. 10 is an exploded perspective view illustrating a portion of a polishing water tank of the system for reducing the lens polishing wastewater according to an embodiment of the present invention.

FIG. 11 is a side view illustrating a portion of a polishing water tank of the system for reducing the lens polishing wastewater according to an embodiment of the present invention.

FIG. 12 is a perspective view illustrating a diffuser of the system for reducing the lens polishing wastewater according to an embodiment of the present invention.

### BEST MODE FOR CARRYING OUT THE INVENTION

Hereinafter, embodiments of the present invention will be described in more detail with reference to the accompanying drawings.

Unless otherwise defined, all terms (including technical and scientific terms) used herein have the same meaning as generally understood by those skilled in the art. Terms as defined in a commonly used dictionary should be construed as having the same meaning as in an associated technical context, and unless defined apparently in the description, the terms are not ideally or excessively construed as having formal meaning.

Moreover, detailed descriptions related to well-known functions or configurations will be ruled out in order not to unnecessarily obscure subject matters of the present invention.

In addition, the sizes of the elements and the relative sizes between elements may be exaggerated for further understanding of the present invention

As used herein, the term and/or includes any and all combinations of one or more of the associated listed items. When a component include another component, this does not exclude another component, but further include another component unless referred to the contrary.

The meaning of ‘include’ or ‘comprise’ specifies a property, a region, a fixed number, a step, a process, an element and/or a component but does not exclude other properties, regions, fixed numbers, steps, processes, elements and/or components.

In addition, the meaning of the terms “unit” and “unit” means a module form that performs at least one function or a unit or role for processing a certain operation of a system, which is a combination of hardware or software or hardware and software It can be implemented as a device or assembly capable of performing an independent operation or by means of such a method.

Also, terms such as upper, lower, upper, lower, or upper, lower, upper, lower, front and rear, left and right are used for convenience to distinguish the relative positions of each component. For example, the upper side in the drawing may be named or referred to as the upper side and the lower side as the lower side, the longitudinal direction may be named or referred to as the front-back direction, and the width direction may be named or referred to as the left/right direction.

Also, terms such as a first and a second are used to describe various members. That is, the terms such as the first and the second are merely used to distinguish one component from another component. For example, without departing from the scope of the present invention, a first element could be termed a second element, and similarly a second element could be termed a first element.

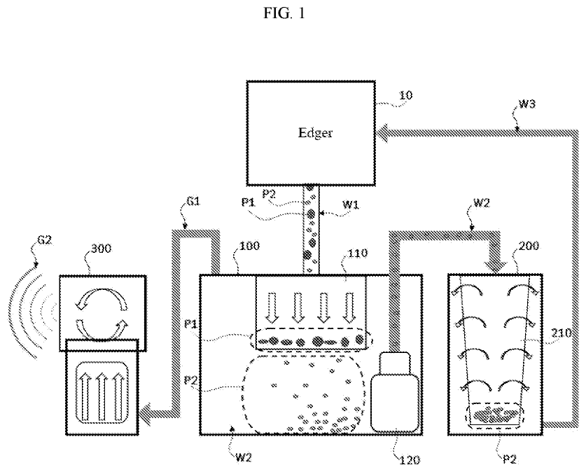

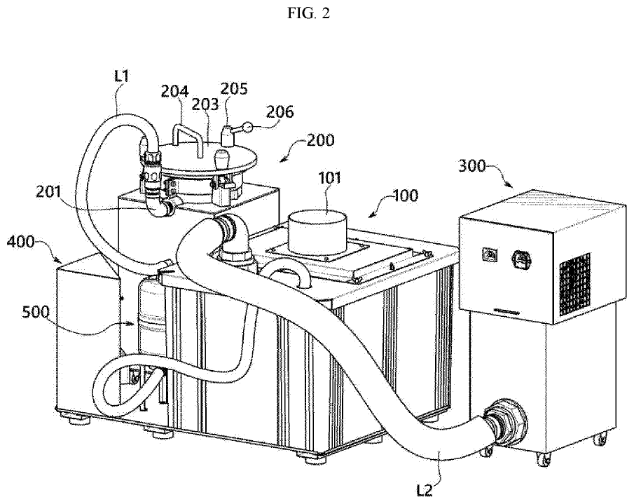

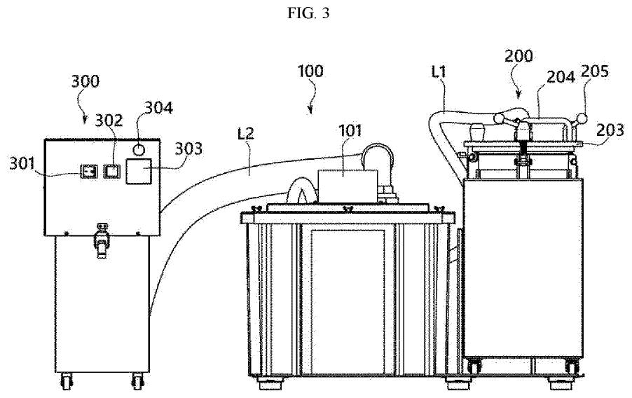

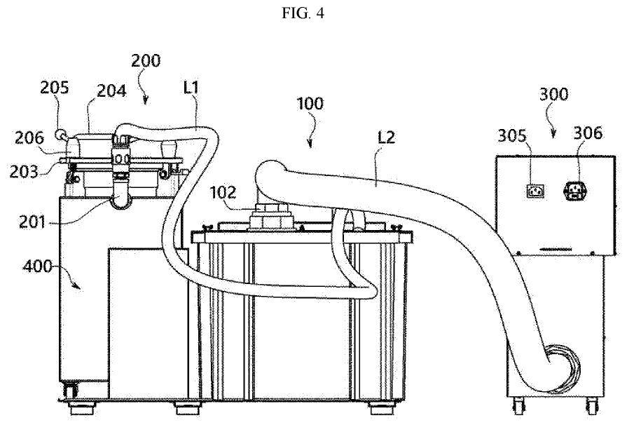

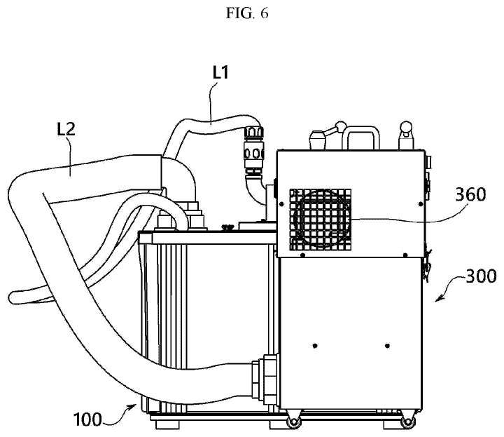

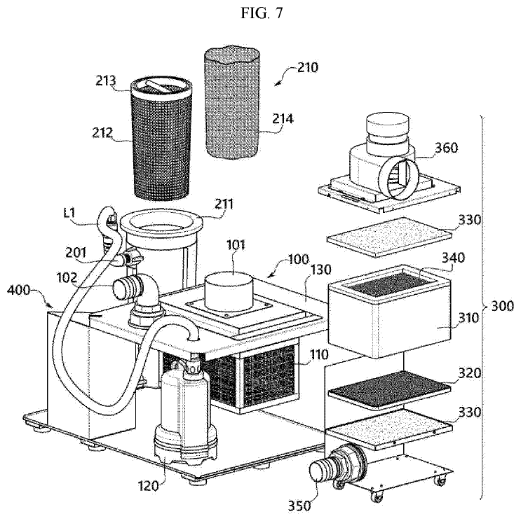

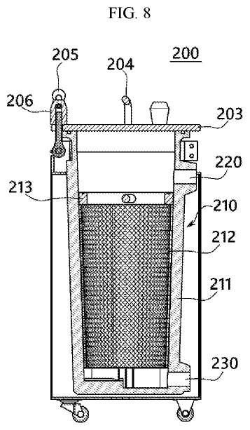

FIG. 1 is a schematic block diagram representing an example of a system for reducing lens polishing wastewater according to an embodiment of the present invention, FIGS. 2 to 6 are views respectively illustrating a perspective view, a front view, a rear view, a right side view, and a left side view of the system for reducing lens polishing wastewater according to an embodiment of the present invention, FIG. 7 is an exploded perspective view of the system for reducing lens polishing wastewater according to an embodiment of the present invention, and FIG. 8 is a cross-sectional view of the of the system for reducing lens polishing wastewater according to an embodiment of the present invention.

As illustrated in FIGS. 1 to 8, the system for reducing lens polishing wastewater according to the present invention includes: a polishing water tank 100 for storing polishing wastewater (or polishing water at the beginning of an operation of an edger) that is supplied to a lens polishing machine 10 (hereinafter, referred to as an ‘edger’) and pumping the stored polishing wastewater to a filtering apparatus 200 by driving a water pump 120 when receiving a lens polishing process signal from the edger 10; and a filtering apparatus 200 for filtering the polishing wastewater pumped from the polishing water tank 100 and then supplying the filtered polishing wastewater to the edger 10.

Also, the system for reducing lens polishing wastewater according to the present invention may further include a deodorizing apparatus 300 for suctioning and filtering smoke (air containing ultrafine particles emitting malodor while diffused in the air) generated when a lens polishing process is performed in the edger 10 and discharging clean air from which the odor is absorbed and removed.

The deodorizing apparatus 300 collects air G1 introduced together with polishing wastewater W1 collected from the edger 10 to the polishing water tank 100 through a suction pipe connection part 350 and discharges clean air G2 after filtering ultrafine particles including odor and carcinogenic or a suspected substances.

Thus, a reduction cycle, in which the polishing wastewater W1 of the edger 10 generated when the lens polishing process is performed by using polishing water supplied from the filtering apparatus 200 is collected to the polishing water tank 100, and the polishing wastewater W1 is pumped to the filtering apparatus 200 by the water pump 120 and the filtered polishing wastewater W1 is supplied as the polishing water to the edger 10 again, is formed.

Also, the air G1 in which the ultrafine particles including the odor and the carcinogenic or suspected substances generated when the lens polishing process is performed in the edger 10 may be filtered in the deodorizing apparatus 300 and then discharged as the clean air G2.

That is, the polishing wastewater W1 in which lens particles P1 and fine lens particles P2 generated when the lens polishing process is performed in the edger 10 is collected in the polishing water tank 100 through a edger connection part 101 disposed below edger 10 due to a self-weight thereof.

The edger connection part 101 has a large diameter so as to be directly applied to various edgers 10 regardless of shapes and kinds of the edgers 10.

A filter net 110 for firstly filtering the lens particles P1 having a relatively large particle size and contained in the polishing wastewater W1 is disposed at an upper portion of the polishing water tank 100.

Since the filter net 110 that is a mesh filter with 20 to 30 meshes filters the lens particles P1 having relatively large particle sizes and a lens or a lens fixing chuck that is able to be dropped to the polishing water tank 100 during or before and after processing, a filtering function thereof is not degraded by clogging of holes for a short time, and primary filtering for the polishing wastewater W1 is smoothly performed for a long time. Thus, the filter net 110 is not required to be frequently replaced or cleaned.

Polishing wastewater W2 collected in the polishing water tank 100 after the lens particles P1 is filtered by the filter net 110 contains the fine lens particles P2 that cause a serious pollution in the ecosystem.

The polishing wastewater W2 collected in the polishing water tank 100 is pumped to the filtering apparatus 200 by the water pump 120 together with the fine lens particles P2.

When the system for reducing the polishing wastewater is initially driven, a proper amount of polishing water to be supplied to the edger 10 is stored in the polishing water tank 100 for the lens polishing process. When the system is operated continuously for a predetermined time, the storage amount of the polishing water decreases as the polishing water is removed together with a dummy (hereinafter, referred to as ‘sludge’) of the fine lens particles P2 filtered by the filtering apparatus 200 simultaneously with natural evaporation.

Thus, the polishing water is required to be added for the proper storage amount.

To this end, the polishing water tank 100 may be made of a transparent material to check the storage amount from the outside, and a scale for displaying a minimum storage amount by which the polishing water is necessarily added may be formed on a side surface of the polishing water tank 100 to maintain the proper storage amount of the polishing water required for the lens polishing process.

Also, a scale for displaying a maximum storage amount in addition to the minimum storage amount may be formed because an extra space is needed at an upper portion of the polishing water tank 100 due to bubbles generated while the edger 10 polishes the lens.

Thus, when the storage amount of the polishing water tank 100 decreases or is expected to decrease below the minimum storage amount, the polishing water is supplied through the edger 10 or added through a tank cover 130 of the polishing water tank 100 to be equal to or less than the maximum storage amount.

Since the fine lens particles P2 are attached to the bubbles generated while the edger 10 polishes the lens, the fine lens particles P2 attached to the bubbles are not precipitated into the polishing wastewater W2 when the bubbles are not removed.

Thus, a defoamer may be inputted to the edger 10 or the polishing water tank 100 to suppress or remove the bubbles generated while the edger 10 polishes the lens.

The defoamer may further include a component having a deodorizing function in addition to a defoaming function.

The filtering apparatus 200 receives the polishing wastewater W2 from the polishing water tank 100 to separate the fine lens particles P2 mixed in the polishing wastewater W2 and stack the separated fine lens particles P2 on the ground, and then supplies reduced water W3 from which the fine lens particles P2 are filtered to the edger 10 as the polishing water.

Here, as a plurality of filtering apparatuses 200 are connected in parallel to each other through valves, although sludge is fully filled in a non-woven bag 214 of one filtering apparatus 200, another filtering apparatus may be consecutively used.

As illustrated in FIG. 8, the polishing wastewater W2 introduced to an upper inlet 220 through a polishing wastewater pipe connection part 201 of the filtering apparatus 200 is pressed by a pressure of the water pump 120 to form a rotary flow above a filter 210 and moves downward to generate a centrifugal force. Accordingly, the fine lens particles P2 mixed in the polishing wastewater W2 are naturally separated from the polishing wastewater W2 and stacked below the filter 210, and the reduced water W3 separated from the fine lens particles P2 is discharged to a low discharge hole 230 through a bottom surface of the filter 210 and then supplied to the edger 10 through a polishing water supply line connected to a reduced water pipe connection part 202.

Here, the filter 210 to which a cyclone principle is applied to separate the fine lens particles P2 by using a mechanical centrifugal includes a casing 211, a mesh net 212, and a non-woven bag 214 to realize the cyclone principle.

The casing 211 may have a tapered shape having a diameter that gradually decreases in a direction from an upper portion to a lower portion thereof so as to form a rotary flow while the polishing wastewater pumped by the water pump 120 is introduced from the upper portion and discharged to the lower portion.

The mesh net 212 has an inverted truncated cone shape such that an outer circumferential surface thereof closely contacts an inner circumferential surface of the casing 211, and a plurality of valleys are obliquely or radially formed in an inner circumferential surface thereof to maximize the cyclone phenomenon.

Here, the mesh net 212 may be cut into a sector shape or a trapezoid shape and then rolled up in the form of a circle to be inserted into the casing 211 so that the mesh net 212 closely contacts an inner wall of the casing 211.

The non-woven bag 214 is mounted into the mesh net 212 while an inlet thereof is fixed to an upper portion of the casing 211 in order to easily remove the fine lens particles P2 from the filtering apparatus 200 when the proper amount of the fine lens particles P2 separated from the polishing wastewater W2 is stacked.

An outer circumferential surface of the non-woven bag 214 closely contacts an inner circumferential surface of the mesh net 212 by the polishing wastewater that is discharged downward while forming the rotary flow at an upper portion of the casing 211, and the polishing water from which fine particles contained in the polishing wastewater are removed is discharged.

The non-woven bag 214 is fixed while the inlet is opened by a fixing ring 213 that is forcedly inserted to the upper portion of the casing 211 so as to store the polishing wastewater W2 forming the rotary flow and the fine lens particles P2 separated from the polishing wastewater W2 and stacked below the filter 210.

Thus, the sludge of the fine lens particles P2 stacked in the non-woven bag 214 may be easily and smoothly removed from the filtering apparatus 200 by pulling out the non-woven bag 214 when the filtering apparatus 200 is stopped.

That is, although the non-woven bag 214 has a rough texture through which the fine lens particles P2 are smoothly transmitted, the sludge of the fine lens particles P2 stacked on the ground through the cyclone principle is remained in the non-woven bag 214, and only the reduced water W3 from which the fine lens particles P2 are separated is transmitted through the non-woven bag 214.

Since a portion of the polishing wastewater W2 is remained in the sludge stored in the non-woven bag 214, the sludge may be solidified by putting an absorbent coagulant that solidifies the polishing wastewater W2 by absorbing moisture of the remained polishing wastewater W2 and then easily separated and put into a volume-based garbage bag for disposal.

A pressure lid 203, a locking arm 205 for fixing the pressure lid 203, and a locking hook 206 may be provided to an upper portion of the filtering apparatus 200 because the polishing wastewater W2 in a state of being pressed by a transfer pressure of the water pump 120 is introduced to the upper inlet 220.

Also, a handle 204 for opening and closing may be formed on the pressure lid 203.

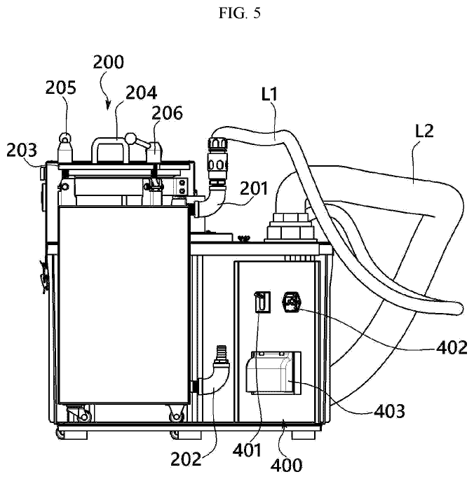

A power supply 400 for supplying a power for overall operation of the system for reducing the lens polishing wastewater is indicated by reference numeral 400 in FIG. 5.

The power supply 400 includes: an edger conjunction outlet 402 that is connected with a power plug applied from the edger 10 to supply and block a power in conjunction with on/off operations of the edger 10; a main switch 401 for turning on/off the power applied to the system for reducing the lens polishing wastewater according to the present invention; and a waterproof outlet 403 connected with each of a power plug of the water pump 120 of the polishing water tank 100 and a power plug of the deodorizing apparatus 300 to supply a power.

Here, the main switch 401 may be an earth leakage circuit breaker having an electric leakage protection function.

Thus, when the edger 10 is operated in a state in which the main switch 401 is turned on, a power is supplied to the system through the edger conjunction outlet 402, and a power is supplied to the water pump 120 and the deodorizing apparatus 300 through the waterproof outlet 403.

Also, control units for controlling operations are provided on a front surface of the deodorizing apparatus 300. The control units may include: a driving switch 301 for turning on/off the deodorizing apparatus 300; a mode switch 302 for driving only the deodorizing apparatus 300 independently from or in conjunction with the system; a timer 303 for setting a driving time of the deodorizing apparatus 300 to operate the deodorizing apparatus 300 for a predetermined time although the edger 10 is turned off when the mode switch 302 is in a conjunction mode; and a driving lamp 304 for displaying whether the deodorizing apparatus 300 is driven.

Also, a conjunction power outlet 305 connected with the waterproof outlet 403 of the power supply 400 for receiving a driving power in conjunction with the edger 10 and a direct power outlet 306 for directly receiving an external power to drive the deodorizing apparatus 300 independently from the driving of the edger 10.

Hereinafter, the conjunction mode will be described as an example. The deodorizing apparatus 300 suctions air G1 introduced together with the polishing wastewater W1 collected to the polishing water tank 100 from the edger 10 as a fan 360 is driven.

While the suctioned air G1 moves from a lower portion to an upper portion of the deodorizing apparatus 300, fine particles in the air are firstly filtered by a cabin filter 320, malodorous substances are secondarily filtered by a carbon filter 310 formed of carbon particles, and ultrafine particles that are carcinogenic or suspected substances are thirdly and lastly filtered by a HEPA filter. Thus, clean air G2 is discharged through an exhaust hole.

The deodorizing apparatus 300 filters fine particles, malodor, and ultrafine particles including the carcinogenic or suspected substances through three filtering processes and discharges the clean air G2.

Here, an inexpensive non-woven sheet 340 may be further provided between the carbon filter 310 and the HEPA filter 330 to block carbon particles introduced from the carbon filter 310 to the expensive HEPA filter 330.

### MODE FOR CARRYING OUT THE INVENTION

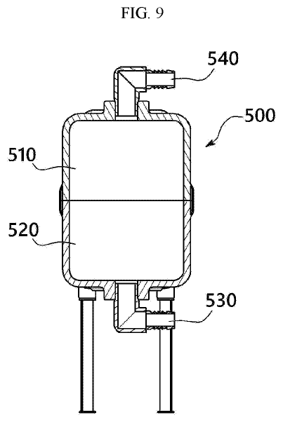

FIG. 9 is a cross-sectional view illustrating an air pocket of the system for reducing the lens polishing wastewater according to an embodiment of the present invention. An air pocket 500 is disposed in the middle of a polishing wastewater pipe L1 connected from the water pump 120 to the filtering apparatus 200.

Since the polishing wastewater W2 fed from the water pump 120 to the filtering apparatus 200 through the polishing wastewater pipe L1 is introduced through the upper inlet 220 of the filtering apparatus 200 by a feed pressure of the water pump 120, and the reduced water W3 is discharged through the low discharge hole 230, an inner pressure of the filtering apparatus 200 is higher than that of the water pump 120 when driving of the water pump 120 is stopped, and air is filled into the water pump 120 as air in the filtering apparatus 200 flows backward through the polishing wastewater pipe L1.

Since the water pump 120 is not operated when air is filled in the water pump 120, the air is required to evacuate the air from the polishing wastewater pipe L1 before the edger 10 is operated.

To this end, the air pocket 500 is provided in the middle of the polishing wastewater pipe L1 connected from the water pump 120 to the filtering apparatus 200.

A water pump connected part 530 connected with the water pump 120 is formed at a lower portion of the air pocket 500, a filtering apparatus connected part 540 connected with the filtering apparatus 200 is formed at an upper portion of the air pocket 500, and an air buffer 510 and a polishing wastewater buffer 520 are formed in the air pocket 500.

Thus, when the water pump 120 is stopped, air in the filtering apparatus 200, which has a relatively high pressure, flows through the polishing wastewater pipe L1 and is introduced to the air buffer 510 through the filtering apparatus connected part 540.

Here, the pressure of the air flowing backward may decrease when the air meets the air buffer 510 having a space wider than that of the polishing wastewater pipe L1, and this may cause loss of a force of pushing the polishing wastewater W2 filled in the air pocket 500 to the water pump 120. Thus, the water pump 120 may be normally operated when the edger 10 is driven again.

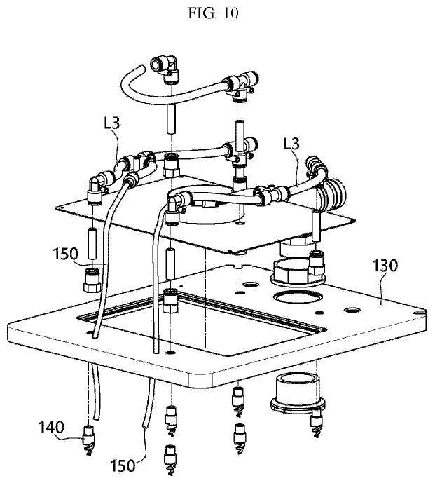

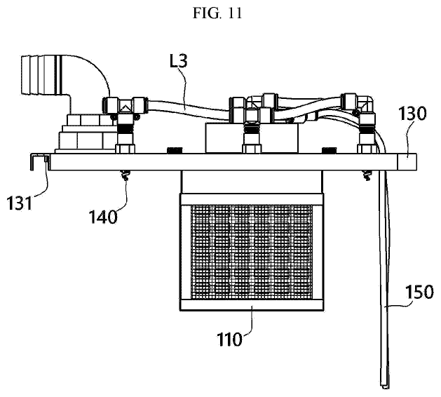

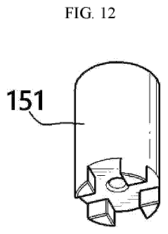

FIG. 10 is an exploded perspective view illustrating a portion of the polishing water tank of the system for reducing the lens polishing wastewater according to an embodiment of the present invention, FIG. 11 is a side view illustrating a portion of the polishing water tank of the system for reducing the lens polishing wastewater according to an embodiment of the present invention, and FIG. 12 is a diffuser of the polishing water tank of the system for reducing the lens polishing wastewater according to an embodiment of the present invention.

As illustrated in FIGS. 10 and 11, a branch pipe L3 branched from the polishing wastewater pipe L1 for supplying the polishing wastewater pumped by the water pump 120 to the filtering apparatus 200 is installed on the tank cover 130 of the polishing water tank 100.

Also, a plurality of spray nozzles 140 for spraying the polishing wastewater supplied through the branch pipe L3 into the polishing water tank 100 from an upper portion thereof are mounted to an end of the branch pipe L2 in order to further completely remove bubbles in the polishing water tank 100.

Here, the spray nozzle 140 may be a spiral cone spray nozzle for generating diffusion spraying of the polishing wastewater.

Also, a blocking wall 131 for preventing the polishing wastewater sprayed from the spray nozzle 140 from being splashed and leaked to the outside is formed on a bottom surface of the tank cover 130.

Also, a plurality of tubes 150 for supplying the polishing wastewater to an inner floor of the polishing water tank 100 are connected with the branch pipe L3 so as to generate a flow of the polishing wastewater in the polishing water tank 100.

That is, the tube 150 may cause a natural flow of the polishing wastewater to prevent the fine lens particles P2 mixed in the polishing wastewater in the polishing water tank 100 from being precipitated to a floor or a corner spaced apart from the water pump 120.

Here, as illustrated in FIG. 12, a diffuser 151 for diffusing and spraying the polishing wastewater discharged from the tube 150 in all directions may be mounted to the end of he tube 150.

Through the above-described configuration, the filtering apparatus for lens polishing wastewater and the system for reducing the polishing wastewater including the same according to the present invention may filter and circulate the polishing wastewater to reuse the polishing wastewater as the polishing water without frequent replacement or cleaning of the filter for filtering the fine lens particles for a long time, and effectively filter and remove the fine particles such as lens particles and fine lens particles from the polishing wastewater.

Also, a clean working environment may be maintained by removing the malodor and the carcinogenic or suspected substances generated during the polishing of the lens.

That is, the filter net 110 in the polishing water tank 100 separates the lens particles P1 having a relatively large particle size and the lens or the lens fixing chuck that is able to be dropped to the polishing water tank 100 during or before and after processing, and the water pump 120 moves small sludge and the polishing water. Malodor generated in this process may be deodorized by the deodorizing apparatus 300.

Also, the filter 210 of the filtering apparatus 200 may separate and stack the fine lens particles P2 contained in the polishing wastewater and remove the fine lens particles P2 as sludge to purify the polishing wastewater and reuse the polishing wastewater as the polishing water.

In addition, since a plurality of lenses are consecutively processed, and the polishing wastewater is continuously circulated instead of being accumulated, a clean edger working environment may be provided.

Also, the sludge may be simply and separately discharged by completely removing moisture contained in the filtered sludge. The filtering apparatus for lens polishing wastewater and the system for reducing the polishing wastewater including the same according to the present invention may be used in all sorts of lens polishing machines regardless of the kinds and shapes of the lens polishing machines.

The above-disclosed subject matter is to be considered illustrative, and not restrictive, and the appended claims are intended to cover all such modifications, enhancements, and other embodiments, which fall within the true spirit and scope of the present invention.

Thus, to the maximum extent allowed by law, the scope of the present invention is to be determined by the broadest permissible interpretation of the following claims and their equivalents, and shall not be restricted or limited by the foregoing detailed description.

1. A filtering apparatus for lens polishing wastewater, comprising:
a casing having a tapered vertical cross-section having a diameter that gradually decreases in a direction from an upper portion to a lower portion thereof so as to form a rotary flow while polishing wastewater pumped by a water pump is introduced from the upper portion and discharged to the lower portion;
a mesh net inserted into the casing in such a manner that an outer surface thereof closely contacts an inner wall of the casing, and a plurality of valleys are obliquely or radially formed in an inner surface thereof, the mesh net configured to accelerate formation of the rotary flow so that fine lens particles is separated from the polishing wastewater and stacked, in the form of sludge, on a bottom surface of the casing; and
a non-woven bag that is made of non-woven fabric having a structure through which the fine lens particles pass, the non-woven bag mounted to the inside of the mesh net in such a manner that an inlet thereof is fixed to the upper portion of the casing, and an outer surface thereof closely contacts the inner surface of the mesh net by the polishing wastewater discharged downward while forming the rotary flow at the upper portion of the casing, so that the bag in which the sludge is stacked from a bottom surface thereof is discharged to the upper portion of the casing, and reduced water from which the fine lens particles are removed is discharged to the lower portion of the casing.

2. A system for reducing polishing wastewater, comprising:
the filtering apparatus of claim 1; and
a polishing water tank configured to store polishing wastewater discharged from a lens polishing machine and pump the stored polishing wastewater by a water pump operated according to driving of the lens polishing machine to the filtering apparatus,
wherein the system supplies the reduced water discharged from the filtering apparatus to the lens polishing machine.

3. The system of claim 2, further comprising a filter net disposed in the polishing water tank and configured to prevent lens particles having a relatively large particle size and contained in the polishing wastewater discharged from the lens polishing machine and a lens or a lens fixing chuck that is dropped to the polishing water tank during or before and after processing from being introduced into the polishing water tank.

4. The system of claim 2, further comprising:
a branch pipe that is branched from a polishing wastewater pipe installed to supply the polishing wastewater pumped by the water pump to the filtering apparatus and installed on a tank cover of the polishing water tank; and
a plurality of spray nozzles mounted to an end of the branch pipe to spray the polishing wastewater supplied through the branch pipe into the polishing water tank from an upper portion thereof in order to remove bubbles disposed at the upper portion of the polishing water tank.

5. The system of claim 4, further comprising a plurality of tubes connected with the branch pipe and configured to supply the polishing wastewater to an inner floor of the polishing water tank so as to generate a flow of the polishing wastewater in the polishing water tank.

6. The system of claim 5, wherein a diffuser is mounted to an end of the tube in order to diffuse the polishing wastewater in all directions.

7. The system of claim 4, wherein a blocking wall for preventing the polishing wastewater sprayed from the spray nozzle from being splashed and leaked to the outside is formed on a bottom surface of the tank cover.

8. The system of claim 2, further comprising a deodorizing apparatus configured to suction air in the polishing water tank and discharge the air to the outside after filtering fine particles and ultrafine particles in the air.

9. The system of claim 2, further comprising an air pocket disposed between the water pump and the filtering apparatus to reduce a pressure of air flowing backward to the water pump from the filtering apparatus due to pressure change when the water pump is stopped.
